(19) United States
(12) Patent Application Publication
(10) Pub. No.: US 2013/0102953 A1
(43) Pub. Date: Apr. 25, 2013

(54) Nano Mixed Metal Oxide Thin Film Photocatalyst Consisting Of Titanium, Indium and Tin

(71) Applicants: Aryasomayajula Subrahmanyam, Chennai (IN); Paul J Tangaraj Ramesh, Chennai (IN)

(72) Inventors: Aryasomayajula Subrahmanyam, Chennai (IN); Paul J Tangaraj Ramesh, Chennai (IN)

(73) Assignee: HCL TECHNOLOGIES LTD., Chennai (IN)

(21) Appl. No.: 13/806,443

(22) PCT Filed: Jun. 23, 2010
(86) PCT No.: PCT/IB10/52853

§ 371 Date: Dec. 21, 2012

(51) Int. Cl. A61K 41/00; B01J 37/34; A61N 5/06; B01J 35/00

(52) U.S. Cl. CPC A61K 41/00; B01J 35/004; B01J 37/348; A61N 5/062
USPC 604/20; 502/7; 502/5; 424/650

(57) ABSTRACT

The present invention relates to a novel photocatalyst comprising Nano mixed metal oxides of titanium, Indium and tin as a thin film with nano sized grains, method of its preparation and applications. The photocatalyst disclosed herein can be used in oxygenation of human/mammalian blood along with all other applications of photocatalysts. A photocatalytic oxygenator for the oxygenation derives oxygen from the water content of mammalian blood. The photocatalyst disclosed herein can also be used for effluent treatments along with all other applications associated with photocatalysts.

### FIELD OF INVENTION

The present invention is directed to a novel photocatalyst comprising nano metal oxides of titanium, Indium and tin as a thin film with nano sized grains and the method of its preparation. The photocatalyst disclosed herein can be used in a photocatalytic oxygenation from the water content of mammalian blood. Said photocatalytic oxygenator is used for performing extracorporeal oxygenation of patient's blood for various chronic and acute pulmonary disorders and to generate oxygen either in-vitro or in-vivo in mammalian blood. The photocatalyst disclosed herein along with all other applications associated with photocatalysts can also be used effectively in the following fields/areas:
Effluent treatments: Air (removal/oxidation of CO and NOX and water purification organic pollutants (pollutant water from the textile and leather and similar industries), Sterilization of medical equipment and hospital floors and walls, Antifogging and self cleaning of facades and window panes in buildings (this application includes the hydrophillicity property of photocatalysts).

### BACKGROUND OF THE INVENTION

Oxygen in continual supply is essential for human life. Gas exchange with the atmosphere accepting oxygen from the atmosphere and excreting carbodioxide into it is the lungs chief function. The details of the structure of the lungs can be found in any human physiological books. The lungs consists of alveoli (or air sacs) measuring to about 70 square meters of area. The total volume of the lung varies between 3.6 to 9.4 liters in adult men and 2.5 to 6.9 liters in adult women. The volume of the pulmonary capillary circulation is about 150 ml which is spread over a surface area of 750 square feet. The blood and air are brought together closely by a membrane of thickness ˜1.0 micron meter in alveoli. The difference in gas pressure of oxygen in alveoli and the blood is responsible for the exchange of oxygen from alveoli into blood and carbon dioxidfe from blood to alveoli (for exhaling CO2 into atmosphere). Hemoglobin, the iron-containing pigment of red blood cells carriers oxygen from the lungs to the tissues.

It is estimated that the number of deaths in USA from all lung disease is estimated approximately at 250,000 (of which 150,000 relate to acute, potentially reversible respiratory failure and 100,000 related to the chronic irreversible respiratory failure). The rate of death related to chronic pulmonary lung disease (CPLD) has increased by 54%. Lung disease also represents one of the major causes of infant mortality.

Hypoxia indicates the situation where tissues are unable to undergo normal oxidative processes because of a failure in the supply or utilization of oxygen. the causes of hypoxia can be grouped into four categories:
1. Hypoxic hypoxia: Hypoxic hypoxia is defined as an inadequate partial pressure of oxygen (PO2) in arterial blood. This can result from an inadequate PO2in the inspired air (such as at altitude), major hypoventilation (from central or periferal causes) or from inadequate alveolar-capillary transfer.
2. Anemic hypoxia: the oxygen content of arterial blood is almost all bound to hemoglobin (Hb). In the presence of severe anemia, the oxygen content will therefore fall in proportion to the reduction in Hb concentration, even though the PO2 is normal. The normal compensatory mechanism to restore oxygen delivery is an increase in cardiac output, but when this can no longer be sustained tissue hypoxia results. Conditions in which Hb is rendered ineffective in binding oxygen, such as carbon monoxide poisoning, produce a reduction in oxygen carriage similar to anemia.
3. Circulating or stagnant hypoxia: if circulatory failure occurs, even though the oxygen content of arterial blood may be adequate, delivery to the tissues is not. Initially tissue oxygenation is maintained by increasing the degree of oxygen extraction from the blood, but as tissue perfusion worsens this becomes insufficient and tissue hypoxia develops.
4. Histotoxic hypoxia: this describes where cellular metabolic processes are impaired to prevent oxygen utilization by the cells, even though oxygen delivery to the tissues is normal. The best-known cause of histotoxic hypoxia is cyanide poisoning, which inhibits cytochrome oxidase.

Oxygen therapies: Photo-catalyst is the substances that regulate light-catalysed reactions. There have been numerous efforts in the past 40 years to achieve artificial lung function.

U.S. Pat. No. 4,061,55 discloses a water splitting apparatus with grated nickel oxide as cathode and photocatalytic N-type semiconductor (Rutile-TiO2) as anode and NaOH dissolved water as electrolyte. The electrodes are biased using a potential developed across Solar cell. On biasing junction is created in anode/electrolyte interface. So when light is shine on the anode, electron-hole pair is generated with hole migrating towards anode/electrolyte electrolyte and electrons move towards cathode. The holes present in anode/electrolyte interface reacts with hydroxyl ions present in the electrolyte and after several complex reactions and products forms oxygen and water. The generated electrons move to cathode under the influence of biasing voltage and in the cathode/electrolyte interface the electrons react with the water to form hydrogen and hydroxyl ions (the process is rather complex). Thus hydrogen is evolved in the cathode and oxygen is evolved in anode using photocatalysis of water.

U.S. Pat. No. 4,793,910 discloses a photoelectrochemical cell used for photolysis of water to produce H2 and O2 without external bias. The cell consist of TiO2/Pt multi-electrode and produces H2 and O2. The reproducible bipolar TiO2/Pt photoelectrodes were fabricated by oxidation (spark anodization) of thin Ti foil pre-deposited with Platinum. While water comes in contact with TiO2/Pt electrode, the photogenerated holes (h+) move to TiO2 interface to cause oxidation of hydroxyl ions to oxygen and water and electrons (e−) moves to Pt interface to create reduction of water to hydroxyl ions and hydrogen. Thus in the presence of UV light, water splitting takes place and H2 and O2 were produced in the molar ration of 2.4:1.

U.S. Pat. No. 5,779,912 disclose an apparatus to clean up air and water from organic contaminants. UV illuminated porous semiconductor (TiO2) film in the presence of powerful oxidants like ozone, hydrogen peroxide or oxygen dissolves the organic contaminants present in water and air into its constituent oxides and water. An UV source of wavelengths 220-280 nm were used in the apparatus for efficient oxidizing. The photocatalytic reactor oxidizes a variety of organic contaminants at ambient temperature and low pressures. The list contaminants the apparatus removed from water include organic solvents like acetone, chlorobenzene, cresols, formaldehyde, hydrazine, isoproponal, methyl ethyl ketones naphthalene, phenols, toluene, pesticides and herbicides, oil spills. Explosives like TNT, RDX, were also removed from air.

U.S. application No. 11/441,547 disclose a system for Ultraviolet blood irradiation. The device consists of UV source providing predetermined wavelength of radiation, an exposure chamber for exposing blood to UV light, an exposure chamber for exposing a predetermined volume of blood to radiation, A peristaltic pump for pumping blood from veins to the system as well as from the system in to artery and a shutter assembly to control the irradiation period.

Another approach to artificial lung function, extracorporeal membrane oxygenation (ECMO), constitutes a mechanism for prolonged pulmonary bypass, which has been developed and optimized over several decades but has limited clinical utility today as a state-of-the-art artificial lung. The ECMO system includes an extra-corporeal pump and membrane system that performs a gas transfer across membranes. Despite the numerous advances in the implementation of ECMO over the years, its core technology is unchanged and continues to face severe limitations. The limitations of ECMO include the requirement for a large and complex blood pump and oxygenator system; the necessity for a surgical procedure for cannulation; the need for systemic anticoagulation; a high rate of complications, including bleeding and infection; protein adsorption and platelet adhesion on the surface of oxygenator membranes; labor intensive implementation; and exceedingly high cost. As a result of these limitations, ECMO has become limited in its utility to select cases of neonatal respiratory failure, where reversibility is considered to be highly likely.

The development of the intravenous membrane oxygenation (IVOX) also represented a natural extension in the artificial lung art, since it was capable of performing intracorporeal gas exchange across an array of hollow fiber membranes situated within the vena cava but did not require any form of blood pump. IVOX (Cardiopulmonics Inc., Salt Lake City, Utah) is an intravenous oxygenator that uses a gradient-driven gas exchange across a gas-permeable, liquid-impermeable membrane. Intravascular devices such as IVOX are surface area limited; they have a high rate of complications and currently provide inadequate gas exchange to function as a bridge to lung transplant or recovery. A further approach to treat lung disease, is through the use of lung transplants, which has currently a 1 year survival around 70%. The major limiting factor to lung transplantation is donor shortage. Only 10% of organ donors are suitable as lung donors and almost 40% of patients on lung transplant lists will die without a donor.

Therefore there is a need of a novel photocatalytic material having an enhanced photocatalytic activity as compared to the known photocatalyst which can be used in various fields like in oxygenators with higher efficacy to provide intermediate to long-term respiratory support for patients suffering from severe pulmonary failure. Also there exists a need for a technology to achieve a sustained gas exchange in the blood, thereby either bypassing the diseased lungs or assisting the lungs without resorting to chronic ventilation, thereby bypassing the diseased lungs without resorting to chronic ventilation, remains the paramount. Also there exists a need for an improved photocatalyst for Effluent treatments for clean environment.

### SUMMARY OF THE INVENTION

The invention is a novel photocatalyst material in thin film form with nano sized grains. The photocatalyst consists of Mixed Metal oxide of Indium, tin and titanium [(In:Sn:Ti) O]. The composition of the thin film material has been evaluated by Energy Dispersive Analysis of X rays (EDX). Further, the present invention provides a method of preparation of the novel mixed metal oxide catalyst comprising DC magnetron sputtering and subsequent annealing.

One object of the present invention is to provide a photocatalytic oxygenator in which dissolved oxygen (DO) is generated directly from the water content of blood of mammalian body through the indirect interaction of ultraviolet (UV) light with a novel photocatalyst. Said oxygenator provides intermediate to long-term respiratory support for patients suffering from severe pulmonary failure and achieves sustained gas exchange in the blood, thereby bypassing the diseased lungs without resorting to chronic ventilation.

Another object of the invention is the use of the novel photocatalyst material in thin film form with nano sized grains disclosed herein along with all other applications associated with photocatalysts can also be used effectively in Effluent treatments preferably for Air (removal of CO and NOX) and water purification organic pollutants (pollutant water from the textile and leather and similar industries), Sterilization of medical equipment and hospital floors and walls, Antifogging and self cleaning of facades and window panes in buildings (this application includes the hydrophillicity property of photocatalysts).

### DETAILED DESCRIPTION

The present invention provides a novel photocatalyst material in thin film form with nano sized grains. . The photocatalyst consists of Mixed Metal oxide of Indium, tin and titanium [(In:Sn:Ti) O]. The atomic percentage of constituents are Indium present in the range of about 3.58-4.80; tin is present in the range of about 0.29-0.32 and Titanium is present in the range of about 0.62-0.72, along with oxygen. The composition of the thin film material has been evaluated by Energy Dispersive Analysis of X rays (EDX). The Photocatalyst has the material consists of tin doped indium oxide (ITO) and titanium dioxide (TiO2). The photo energy required by said photocatalyst is any single or a range of wavelengths in the spectrum 255 nm-1100 nm.

One embodiment of the present invention is to provide a method of preparation of said phototcatalyst. The method of preparation of the novel mixed metal oxide catalyst with nano sized grains consists of deposition of the metal oxides by DC magnetron sputtering technique on a substrate followed by annealing. The new material (NM) is a thin film consisting of mixed metal oxides of titanium, tin, indium. Reactive DC Magnetron sputtering is employed to prepare the thin films. A commercial sputtering unit is employed for the purpose. The sputtering unit consists of a growth chamber, rotary and diffusion pumps for evacuation, vacuum measuring gauges and water coolant feed troughs for the targets and an arc suppression DC Magnetron power supply. The growth chamber should consist of a mechanism to rotate the substrates for uniform coatings. The diffusion pump is supported by the rotary pump in the DC magnetron sputtering unit. Any standard commercial sputtering system can be employed for preparing the mixed metal oxide thin films with suitable growth parameters. The non-limiting representative growth parameters are given under the head ‘sample preparation’. The targets used are commercially available pure (99.9%) metallic Titanium and alloy target of indium and tin (90:10 by weight %). Initially, the growth chamber is evacuated to a base pressure of ˜10⁻⁶ milli bar by a combination of rotary and diffusion pumps. Then pure argon and oxygen gases are introduced into the growth chamber at specified flow rates such that the growth chamber vacuum is at ˜3-4×10⁻³ milli bar. In order to maintain these pressures in the growth chamber, the sputtering system should have a throttle valve. Then the target is powered by a magnetron power supply (similar to Advanced Energy where there is a provision for arc suppression). The thin films are prepared for desired thickness either by employing a thickness monitor or by noting the time of sputtering. All the thin films are prepared at room temperature +300K; however, during sputtering there will be an inherent increase in the temperature at the substrate and it should not exceed more than 80° C. for the complete growth of the thin films. The temperature of deposition should lie in the range 300K to 400 K. The suitable substrates used in the method are quartz, synthetic silicon dioxide, soda lime glass, poly-carbonates and poly imides and any suitable polymers. The most suitable substrates are quartz and synthetic silicon dioxide.

Sample Preparation

The substrates employed were chemically cleaned quartz plates and quartz tubes. The plates were used for characterizing the NM thin films and the tubes were used for the measurement of photocatalytic activity. The dimensions of the quartz plates were: 38 mm(length)×09 mm (breadth)×01 mm (height) and quartz tube had the dimension : 255 mm (length)×19 mm (inner diameter). These substrates were cleaned following the standard cleaning methods: soap wash, acetone wash, chromic acid wash followed by distilled water clean up and dried with nitrogen gas. The cleaned substrates were loaded into the growth chamber.

Initially, tin doped indium oxide (ITO) coatings of desired thickness were carried out; then without breaking vacuum, TiO2 coatings of required thickness were carried out. The non-limiting growth/process parameters employed in the process were as follows:

| PARAMETERS | ITO | | TiO₂ | |
|---|---|---|---|---|
| Initial pressure | 3.2 × 10⁻⁵ | mbar | 3.8 × 10⁻⁵ | mbar |
| Coating Pressure | 3.7 × 10⁻³ | mbar | 6.5 × 10⁻³ | mbar |
| Argon flow rate | 20-24 | Sccm | 30-35 | Sccm |
| Oxygen flow rate | 2.0-9.5 | Sccm | 5-7 | Sccm |
| Power (Advanced Energy | 80 | Watts | 400 | Watts |
| MDX Power supply) | | | | |
| Current as shown byt eh | 0.28 | Amps | 1.16 | Amps |
| DC magnetron power supply | | | | |
| Voltage as shown by the | 338 | Volts | 331 | Volts |
| DC Magnetron power supply | | | | |
| Time of deposition | 60 | Mins | 60-180 | Mins |
| Rotation speed | 10 | Rpm | 10 | Rpm |

Note:
These growth parameters are indicative but not exclusive.

The coated substrates with these thin films are annealed at a temperature ranging between 500° C.-700° C. preferably ˜600±10° C. in ambient air. These are the NM thin films. These mixed metal oxides are characterized for their structural, electrical, optical and photocatalytic properties.

Another embodiment of the present invention is the a photocatalytic oxygenator and a method of oxygenation of blood of mammaliam body comprising the novel photocatalyst layer of mixed metal oxides of titanium, Indium and tin as a thin film with nano sized grains. The atomic percentage of the constituents in said photocatalytic layer is: Indium in the range of about 3.58-4.80; tin in the range of about 0.29-0.32 and Titanium in the range of about 0.62-0.72 (as measured by EDX measurements on the thin films) along with oxygen. The photocatalytic oxygenator comprising the novel photocatalyst layer effectively perform the extrocrporeal oxygenation of patien's blood for various chronic and acute pulmonary disorders and generates oxygen either in-vitro or in-vivo of the blood in mammalian body.

The novel photocatalyst of the present invention is found to be more efficient compared with the conventional titanium dioxide photocatalyst. The novel photocatalyst disclosed herein along with all other applications associated with photocatalysts can also be used effectively in Effluent treatments: Air (removal of CO and NOX and water purification organic pollutants (pollutant water from the textile and leather and similar industries), Sterilization of medical equipment and hospital floors and walls, Antifogging and self cleaning of facades and window panes in buildings (this application includes the hydrophillicity property of photocatalysts).

### EXAMPLES

### Example 1

Method employed for the measurement of the photocatalytic activity of NM to compare it with the photocatalytic activity of the normal titanium dioxide. The photocatalytic activity of these thin films (both plates and tubes) is characterized using the principle of oxidation of organic dyes. Rhodamine B is the dye chosen for the purpose. For tubes, the actual oxygen (dissolved oxygen) produced by photocatalysis is evaluated by using an oxyprobe procured from M/s Ocean optic company (USA).

Photocatalytic activity measurements on plates: The coated plates are placed in a Petridish and it is filled with 0.002 mole % of Rhodamine B solution (10 mg of Rhodamine B is dissolved in 1 litres of pure distilled water). Before the samples are exposed to UV light (254 nm and 365 nm), the optical absorption (optical density) of Rhodamine Red (1.8 ml filled in a quartz cuette) is measured by a double beam optical spectrophotometer (JASCO). When the catalyst is exposed to the UV light, the optical transmission of Rhodamine Red is measured in regular intervals of 30 minutes. The decrease in the optical absorption of Rhodamine Red with time as a result of photocatalytic action (oxidation) is a measure of photocatalytic activity.

The Photon flux for the photocatalytic activity is measured by Actinometry experiments and by power meters.

Photocatalytic activity in Tubes: The tubes are coated with the NM thin film on the outer surface. 180 milli litre volume of 0.002 Mol % Rhodamine Red solution surrounds the tube and it is in close contact with the NM thin film. A UV lamp is inserted into the tube and the lamp illuminates the light from the inner surface into the outer surface. 3.0 ml of Rhodamine Red dye is collected in a quartz cuette periodically (every 30 minutes) with exposure to UV light and the optical absorption of Rhodamine Red dye is measured by a double beam spectrophotometer (JASCO).

### Example 2

Haemocompatibility Study

Preliminary evaluation of the effects of the photocatalytic oxygenator on blood have demonstrated no significant fall in pH, no rise in serum potassium or hematocrit, no RBC hemolysis or platelet aggregation.

1. A photocatalyst comprising mixed metal oxides of titanium, Indium and tin as a thin film with nano sized grains.

2-14. (canceled)

15. The photocatalyst as claimed in claim 1, wherein the atomic percentage of constituents are about 3.58-4.80 Indium, about 0.29-0.32 Tin, and about 0.62-0.72 Titanium, as measured by EDX measurements on the thin films, along with oxygen.

16. The photocatalyst as claimed in claim 1, wherein the photocatalyst consists of tin doped indium oxide (ITO) and titanium dioxide (TiO₂)

17. The photocatalyst as claimed in claim 1, wherein the photo energy required is any single or a range of wavelengths in the spectrum of 255 nm-1100 nm.

18. A method making a photocatalyst consisting of a Titanium, Indium and Tin mixed metal oxide thin film with nano sized grains comprising depositing the metal oxides by DC magnetron sputtering on a substrate followed by annealing.

19. The method as claimed in claim 18, wherein the substrate is quartz, synthetic silicon dioxide, soda lime glass, poly-carbonates, poly imides or a polymer.

20. The method as claimed in claim 19, wherein the substrate is quartz or synthetic silicon dioxide.

21. The method as claimed in claim 18, wherein the depositing of the metal oxides is performed at a temperature in the range of 300K to 400 K.

22. The method as claimed in claim 18, wherein the annealing is performed at a temperature ranging between 500° C.-700° C.

23. The method as claimed in claim 22, wherein the annealing is performed at a temperature of about 600° C.±10° C.

24. The method as claimed in claim 18, wherein the DC magnetron sputtering is performed by a DC magnetron sputtering unit that comprises a diffusion pump supported by rotary pump.

25. A photocatalytic oxygenator comprising a photocatalyst layer that comprises mixed metal oxides of Titanium, Indium and tin as a thin film with nano sized grains.

26. The photocatalytic oxygenator as claimed in claim 25, wherein the atomic percentage of constituents are about 3.58-4.80 Indium, about 0.29-0.32 Tin, and about 0.62-0.72 Titanium, as measured by EDX measurements on the thin films, along with oxygen.

27. A method of using the photocatalytic oxygenator of claim 25 to oxygenate blood comprising:
contacting the photocatalytic oxygenator of claim 25 with blood in the presence of ultra violet light.
